(19) United States
(12) Patent Application Publication
(10) Pub. No.: US 2024/0050255 A1
(43) Pub. Date: Feb. 15, 2024

(54) GASTROINTESTINAL IMPLANT

(71) Applicants: Alexander Butz, Wien (AT); Viktor Grablowitz, Wein (AT)

(72) Inventors: Alexander Butz, Wien (AT); Viktor Grablowitz, Wein (AT)

(21) Appl. No.: 18/268,047

(22) PCT Filed: Dec. 18, 2020
(86) PCT No.: PCT/EP2020/086954

§ 371 Date: Jun. 16, 2023

(51) Int. Cl. A61F 5/00

(52) U.S. Cl. CPC A61F 5/0036; A61F 5/003; A61F 5/0076; A61F 5/0089; A61F 2210/0057; A61F 2220/0008; A61F 2220/0075; A61F 2230/0069; A61F 2250/0007; A61F 2250/0008; A61F 2250/0058; A61F 2250/0014

(57) ABSTRACT

The invention relates to an implant for arranging and expanding in the human gastrointestinal tract for the purpose of lining a section of the gastrointestinal tract in order to prevent the resorption of supplied nourishment in said section, having a first end section (1) which, as a securing section, allows the fixation of the implant in the human stomach/gastrointestinal tract and/or to an applicator (10); a tubular section which adjoins the first end section (1), comprising a longitudinal section (2) which extends in the provided expansion direction, a transition section (3) which adjoins the longitudinal section (2), and an inverted section (4) which runs within the longitudinal section opposite the provided expansion direction; and a second end section (8). According to the invention, a region of the inverted section (4) and a region of the longitudinal section delimit a flow channel (5), via which a fluid (15) blown into the tubular section can penetrate the transition section (3) in order to exert pressure thereon, wherein the inverted section (4) radially delimits a cavity (6) and has a closure section (7) which forms the second end section (8) and axially delimits the cavity (6).

### FIELD OF THE INVENTION

The present invention relates to the field of obesity and the associated secondary diseases such as diabetes mellitus, arterial hypertension and the accompanying increased risks of stroke, heart attack and cancer, but also secondary diseases of the spine and musculoskeletal system.

Many patients are often unable to combat their obesity on their own, for example by eating less or in a healthier way. Moreover, patients are often also unable to get the exercise that is so necessary to combat obesity, because the obesity has often already progressed to such an extent that the above-mentioned secondary diseases of the spine and musculoskeletal system cause such severe pain that it is impossible to think about sufficient exercise.

These patients therefore need additional help to quickly reduce the excess weight to a level where at least pain-free movement is possible again. The accompanying increase in well-being very often motivates patients to make further efforts to reduce their weight, so that this initial help with weight reduction helps patients to achieve further weight reduction on their own or at least to maintain the weight reduced to a level that is less harmful to health.

### PRIOR ART

Various methods are known in medicine to make the described rapid weight reduction possible. For example, it is known to perform stomach reductions or to create gastric bypasses. Since this is a difficult, invasive procedure on the human body, this method is not only technically very demanding but also involves a risk associated with any invasive procedure, including the risk associated with the required longer anesthesia time.

In the search for alternative, less risky methods, the practice has been adopted to position a plastic tube with a securing means (gastrointestinal implant) in the human small intestine, in particular the duodenum, and to partially eliminate the resorption of supplied nourishment in this manner. The supplied nourishment is not resorbed in the region of the plastic tube since the plastic tube lines the intestinal lumen on the inside and thus prevents making contact with the intestinal wall and thus the resorption of nutrients. If the plastic tube is placed appropriately in the small intestine (duodenum), immediately adjacent to the pylorus, the plastic tube also causes the digestive enzymes (bile and pancreatic secretions) to come into contact with the food only after the end of the plastic tube, thereby further limiting food utilization and preventing duodenogastric reflux.

The length of the tube can be individually selected and influences the speed and maximum extent with which the weight reduction should progress. Weight reductions of >70% in one year are possible. In addition, as expected, type II diabetes mellitus is already in remission in 80% of patients after 3 months, as has been found in various studies and is known from bariatric surgery. Arterial hypertension, cardiac arrhythmias, musculoskeletal diseases and other comorbidities also improve significantly after such weight reduction and are even in remission.

It is known to place the gastrointestinal implant endoscopically in its position in the small intestine and also to remove it again from there. Thus, no surgery is required. The procedure is non-invasive and therefore scarless and fully reversible. The application of the implant is performed under sedation or short anesthesia.

In order to be able to perform the endoscopic positioning of the gastrointestinal implant in the small intestine, on the one hand, an applicator is required, which initially protects the gastrointestinal implant during insertion into the small intestine, and then allows expansion of the implant in the gastrointestinal tract and subsequent fixation, and on the other hand, suitable securing means, which allow reliable, substantially immovable fixation of the implant in the gastrointestinal tract.

Such implants and applicators are known, for example, from US 2012/0065571 or WO 2020/141023 A1. These documents both show implants with tubular sections that have elastic annular securing means connected in an end region by means of elastic elements. These securing means are positioned on either side of the pylorus to clamp it. In this manner, the implant can be positionally fixed to the pylorus.

In practice, it has now been found that this type of positional fixation of the implant to the pylorus is advantageous.

However, it has proven difficult to expand the tubular section of the implant appropriately into the intestinal tract after positional fixation to the pylorus such that the intestinal tract is lined by the tubular section over the desired length. The length of the intestinal section to be lined depends on the severity of the obesity and the desired prevention of resorption of nourishment.

In this connection, WO 2020/141023 A1 proposes that the end region of the implant as well as a region of the tubular section be pulled over a cup-shaped end region of the applicator having a holding volume, and that the protruding section be stuffed into the holding volume so that it is arranged therein in a folded manner.

By introducing a fluid, preferably air, into the cup-shaped end region, the tubular implant can be expanded into the small intestine by pushing the section that has been folded (invaginated) into the holding volume back out of the holding volume.

In practice, however, it has been found that stuffing the protruding section into the holding volume results in a random folding of the tubular section of the implant, which, although it is easy to create and improves in principle the possibility of expansion of the tubular section into the intestinal tract over previously known methods, may nevertheless result in incomplete expansions, in particular in the case of longer implants, and expansion over several intestinal convolutions of the intestine and thus lining of the intestine over the medically required length is not possible.

### OBJECT OF THE INVENTION

It is therefore the object of the invention to provide a generic implant, the expandability of which is improved over previously known generic implants and which allows complete expansion in the intestinal tract regardless of its length as well as regardless of the complexity of the intestinal convolutions.

### SUMMARY OF THE INVENTION

According to the invention, this object is achieved with an implant for arranging and expanding in the human intestinal tract for the purpose of lining a section of the intestinal tract to prevent the resorption of supplied nourishment in this section, having a first end section which, as a securing section, allows the fixation of the implant in the human gastrointestinal tract and/or to an applicator, and a tubular section adjoining the first end section and comprising a longitudinal section extending in the intended direction of expansion, as well as a transition section adjoining the longitudinal section, and an adjoining inverted section which runs within the longitudinal section in the opposite direction to the intended expansion direction, as well as a second end section, in that a region of the inverted section and a region of the longitudinal section delimit a flow channel via which a fluid blown into the tubular section can penetrate to the transition section in order to exert pressure thereon, and wherein the inverted section radially delimits a cavity and has a closure section which forms the second end section and axially delimits the cavity.

An implant formed in this manner at the time of insertion into the gastrointestinal tract allows safe and, above all, complete unfolding even over several intestinal convolutions. The design according to the invention allows the inverted section to be successively rolled out of the longitudinal section due to a fluid blown into the tubular section. In this case, for one thing, the transition section but also the closure section forming the second end section each offer an engagement surface for the fluid. The cavity radially delimited by the inverted section on the one hand, and the generally substantially annular flow channel formed between the inverted section and the longitudinal section on the other hand, allow the inverted section to roll out in the intended expansion direction and along the intestinal wall, similar to the running gear of a tank but without rollers, and prevents the inverted section from becoming knotted or blocked. The formation of the two engagement surfaces for the fluid to be blown in also has the effect of equalizing the rolling out of the inverted section and prevents the second end section from being pushed through the cavity before the entire inverted section has been rolled out from the longitudinal section.

It is particularly advantageous if the closure section is formed flat, in particular if the formed surface is normal to the longitudinal axis of the inverted or longitudinal section. A flat formation of the closure section provides a particularly good engagement surface for the fluid to be blown into the tubular section. In this context, flat is understood to mean not only a flat surface but also a curved surface.

An alternative embodiment of the invention provides for the closure section to be created by gathering a region of the inverted section which is spaced apart from the transition section. Gathering in this context is understood to mean the compression of the material of the second end section in the axial direction, whereby irregular buckled and compressed material webs are formed in this region. Such a closure section is easier to create than a flat one.

According to another preferred embodiment of the invention, it is provided that the closure section is arranged within the longitudinal section, thus, it does not overlap with the securing section, whereby the connection of the securing section to an applicator cannot be obstructed by the inverted section.

According to another preferred embodiment of the invention, it is provided that the region of the inverted section delimiting the flow channel comprises means for stiffening at least in sections, preferably in its entirety. Particularly preferably, the means for stiffening are foldings.

It should be noted that, depending on the type and strength of the stiffening, said stiffening can make the formation of a cavity and the radial and one-sided axial delimitation thereof by the inverted section obsolete, without the rolling out process being substantially worsened as a result. In one embodiment of the invention, it can therefore also be provided that in an implant according to the invention for arranging and expanding in the human intestinal tract for the purpose of lining a section of the intestinal tract to prevent the resorption of supplied nourishment in this section, having a first end section which, as a securing section, allows the fixation of the implant in the human gastrointestinal tract and/or to an applicator, and having a tubular section which adjoins the first end section, comprising a longitudinal section which extends in the intended expansion direction of expansion, and a transition section which adjoins the longitudinal section, as well as an inverted section which adjoins the transition section and runs within the longitudinal section in the opposite direction to the intended expansion direction, and having a second end section, wherein a region of the inverted section and a region of the longitudinal section delimit a flow channel via which a fluid blown into the tubular section can penetrate to the transition section in order to exert pressure thereon, and the region of the inverted section delimiting the flow channel has means for stiffening, at least in sections.

In this embodiment, therefore, the formation of a cavity delimited by the inverted section is not mandatory. Of course, this embodiment can be combined with all other embodiments described herein.

However, in an alternative embodiment, the means for stiffening can also be sections with a thicker wall thickness or regions made of a different, stiffer material than the remaining regions of the inverted section.

Regardless of how the stiffening is ultimately implemented, it improves the ability of the implant in terms of rolling out the inverted section from the longitudinal section and also improves the ability of the implant to delimit the cavity radially outwards and the flow channel toward the cavity.

According to another preferred embodiment of the invention, it can be provided that the region of the inverted section delimiting the flow channel loosely comes into contact in sections with the region of the longitudinal section delimiting the flow channel, in particular makes contact with it in a releasable manner. On the one hand, this ensures that the radial expansion of the implant is kept small, and on the other hand, it also ensures that the fluid to be blown in is able to detach the regions from one another and to reach the transition section. It follows from the above that the flow channel can be interrupted where regions of the longitudinal section and regions of the inverted section come into contact with each other, but that such an interruption basically does not exist or exists only temporarily in the operating state during the rolling out of the inverted section due to the blown in fluid, and in any case does not impede the rolling out.

Alternatively, it can also be provided that the region of the inverted section delimiting the flow channel runs spaced apart from the region of the longitudinal section delimiting the flow channel, with the advantage that in this case, the fluid to be blown in does not first have to release any contact between the regions.

This can be achieved by sufficiently stiffening the region of the inverted section delimiting the flow channel while at the same time using a sufficiently strong base material of the implant so that accordingly, the longitudinal section is also dimensionally stable.

According to another preferred embodiment of the invention, it is provided that the inverted section is shorter than the longitudinal section, so that overlapping with the securing section and thus complication of connection to an applicator can be excluded.

According to another alternative embodiment of the invention, it can be provided that the securing section forming the second end section is gathered but open. While during rolling out of the inverted section from the longitudinal section, the opening, due to the gathering, nevertheless sufficiently closes the end section for the purposes of pressure build-up by the fluid to be blown in and forms a corresponding engagement surface for the fluid to be injected, the opening in the fully expanded state of the implant effects that the implant is immediately ready for use and no further measures need to be taken to open the second end section.

According to another alternative embodiment of the invention, it can be provided that the closure section (second end section of the implant) is cup-shaped and/or balloon-shaped and protrudes into or out of the cavity in the intended expansion direction. This alternative embodiment offers advantages in that the cup- or balloon-shaped closure section can be filled by the fluid to be blown in and can thus support the rolling out in the intestinal tract. In contrast to the embodiments without a cup-shaped and/or balloon-shaped auxiliary section, in the embodiment with the auxiliary section, the second end section of the implant leaves the longitudinal section earlier and not as the last region of the inverted section.

### BRIEF DESCRIPTION OF THE FIGURES

The present invention will now be described in more detail with reference to one or more figures representing exemplary embodiments. In the figures:

FIG. 1a shows a first embodiment of an implant according to the invention at the time of insertion into the gastrointestinal tract.

FIG. 1b shows a second embodiment of an implant according to the invention at the time of insertion into the gastrointestinal tract.

FIG. 1c shows a third embodiment of an implant according to the invention at the time of insertion into the gastrointestinal tract.

FIG. 1d shows a fourth embodiment of an implant according to the invention at the time of insertion into the gastrointestinal tract.

FIG. 2 shows the first embodiment of the implant according to the invention during expansion into the gastrointestinal tract.

FIG. 3 shows the first embodiment of an implant according to the invention shortly before completion of the expansion into the gastrointestinal tract.

FIG. 4 shows the first embodiment of an implant according to the invention after complete expansion into the gastrointestinal tract.

FIG. 5 shows the connection of the first embodiment of the implant according to the invention to an applicator.

FIG. 6 shows a sectional view along line AA of FIG. 5.

FIG. 7 shows a sectional view along line BB of FIG. 5.

FIG. 8 shows a sectional view along line CC of FIG. 5.

FIG. 9 shows an alternative connection possibility of the first embodiment of the implant according to the invention to an applicator.

FIG. 10 shows an alternative connection possibility of the fourth embodiment of the implant according to the invention to an applicator.

FIG. 10a shows a detailed view of FIG. 10.

### STRUCTURE OF THE IMPLANT

In the following figures, dimensions and proportions are not to scale. Individual components, sections, etc. may be drawn disproportionately large and/or rather schematically or generalized for reasons of comprehension and visibility.

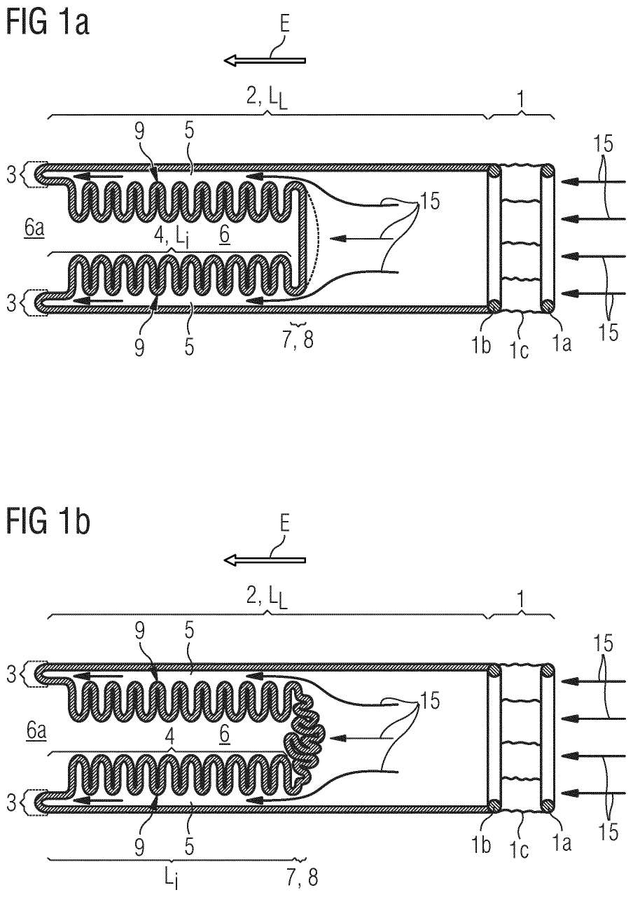

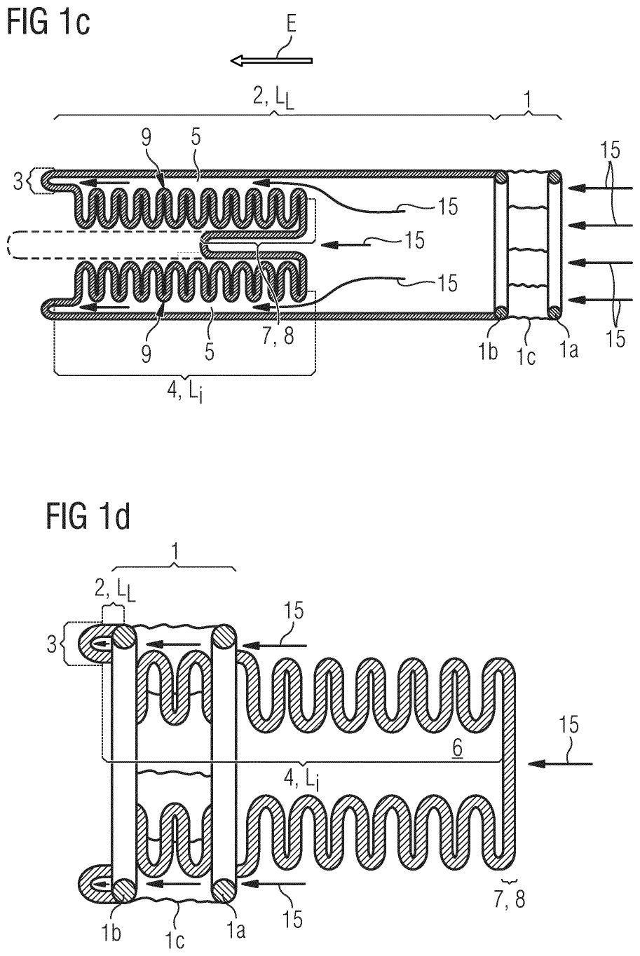

FIG. 1a shows a first embodiment of a gastrointestinal implant according to the invention at the time of insertion into the gastrointestinal tract and before the start of the expansion process in a sectional view. Here, the arrow marked E points in the intended expansion direction of the implant, thus, the direction in which the implant, after being positioned in the human or animal gastrointestinal tract by means of an applicator, expands by filling with a fluid so as to line the intestinal wall.

As will be explained in more detail later, the gastrointestinal implant is configured to be first fixed to an applicator in the form illustrated in FIG. 1a and after being fixed in such a manner, to be conveyed to the correct position in the human gastrointestinal tract.

The gastrointestinal implant is preferably made of silicone and/or fluorosilicone and/or PE or another biocompatible material, from which a wide variety of product forms can be produced using known production methods, and has multiple sections, namely a first, open end section 1, which serves as a securing section for the purpose of securing/fixing both in the gastrointestinal tract and to an applicator. For this purpose, the first end section 1 can be formed by two substantially annular securing elements 1a, 1b, both of which are interconnected by elastic bands 1c, wherein one annular securing element 1b is provided for intestinal-side engagement on the pylorus and the other annular securing element 1a is provided for gastric-side engagement on the pylorus. The elastic bands 1c are dimensioned such that the two annular securing elements 1a, 1b are capable of clamping the pylorus. Typically, the annular securing elements have a diameter between 25 mm and 30 mm. Other diameters are quite conceivable depending on specific anatomical conditions.

However, the two annular securing elements 1a, 1b also serve for securing to the applicator, via which the implant is introduced into the gastrointestinal tract, expanded and fixed, preferably in this order.

Viewed in expansion direction E, the end section 1 is adjoined by a tubular section, the diameter of which is typically between 12 mm and 25 mm. Here, too, however, deviations are quite possible depending on specific anatomical conditions. The tubular section is connected, preferably bonded, to one of the annular securing means, preferably the one provided for securing on the intestinal side, and comprises an open longitudinal section 2, a transition section 3 within which the implant changes its direction of extent, and an inverted section 4 continuing the transition section 3 and extending in the opposite direction to the intended expansion direction E.

The length L_{L} of the longitudinal section 2 at the time of insertion of the implant into the gastrointestinal tract depends on the material of the implant and/or its total length or the desired weight reduction in terms of time and extent. If applicable, the length may additionally also depend on anatomical conditions in the gastrointestinal tract.

In one exemplary embodiment, the length L_{L} of the longitudinal section 2 at the time of insertion into the gastrointestinal tract can be 1 cm to 7 cm, and the fully expanded tubular section typically has an overall length of 70 cm to 300 cm, but this depends on the therapeutic purpose.

In another exemplary embodiment, however, the length L_{L} of the longitudinal section 2 at the time of insertion into the gastrointestinal tract might be only a few mm or less. This is particularly the case if the transition section 3 adjoining the longitudinal section 2 in the expansion direction E is almost immediately adjacent to the end section 1, as illustrated in a fourth embodiment according to FIG. 1d.

At the time of insertion into the gastrointestinal tract, the subsequent section 4 of the implant extends inverted in relation to the longitudinal section 2, thus is invaginated therein and is intended to be rolled out into the intestinal tract in the intended expansion direction E. The transition section 3 connects the longitudinal section 2 to the inverted section 4. The length Lᵢ of the inverted section at the time of insertion into the gastrointestinal tract again depends on the total length of the implant and/or the length L_{L} of the longitudinal section 2 and/or on anatomical conditions of the gastrointestinal tract, but is preferably selected such that it runs completely within the longitudinal section 2, i.e. Lᵢ<L_{L} applies.

However, it is also conceivable that the inverted section 4 is longer than the longitudinal section 2, i.e. Lᵢ>L_{L} applies, and protrudes into or beyond the first end section 1 against the intended expansion direction E, if this is required by particular anatomical conditions and as shown in FIG. 1d.

The longitudinal section 2 and the inverted section 4 delimit, at least along a region, a flow channel 5 between them, via which a fluid blown the into longitudinal section 2, for example through end section 1, can advance to the transition section 3. The region of the inverted section 4 delimiting the flow channel 5 can either make contact in a releasable manner with the region of the longitudinal section 2 delimiting the flow channel 5 (in sections or along the entire region), or these two region can run spaced apart from each other. In the latter case, the flow channel 5 formed by the inversion of the section 4 is of substantially annular shape.

In this case, making contact in a releasable manner is to be understood to mean that by blowing a fluid into the longitudinal section 2, the inverted section 4 and the longitudinal section 2 detach from each other where they possibly come into contact with each other, so that the blown in fluid can reach the transition area 3.

The inverted section 4 radially delimits a cavity 6 that is open in the expansion direction E and has a closure section 7 that axially delimits the open cavity 6 at its end opposite the opening 6a.

The closure section 7 of the inverted section 4 is, at the same time, the second end section 8 of the implant.

It can be formed to be flat, as shown in FIG. 1 with a solid or dashed line. In the case of the dashed line, the closure section is designed to be flat but curved against the intended expansion direction E.

However, the closure section can also be created by gathering, as illustrated in a second embodiment according to FIG. 1b, wherein the gathering is created by compressing the material of the second end section 7 in the axial direction, as a result of which irregularly folded and compressed material webs are formed in this region.

In the event that the closure section 7 is created by gathering, it can also be formed to be open, i.e. that the closure section 7 as second end section 8 of the implant in the rolled out, fully expanded state is already open and no further measures for opening are required, while prior to the complete rolling out of the inverted section 4 from the longitudinal section 2, it closes the inverted section 4 sufficiently for the purposes of pressure build-up by the fluid to be blown into the longitudinal section 2 due to the gathering and therefore axially delimits the cavity 6 and seals it against the intended expansion direction.

Alternatively, according to a third embodiment as illustrated in FIG. 1c, the closure section 7 (second end section 8 of the implant) can be cup-shaped and/or balloon-shaped and protrude into the cavity 6 in the intended expansion direction E (solid line) or protrude out of it through the opening 6a (dashed line) thereof. This alternative embodiment offers advantages in that, due to the cup or balloon shape, the closure section 7 (second end section 8 of the implant) can be filled by the fluid to be blown in and can thus support the rolling out in the intestinal tract. In contrast to the embodiments without a cup-shaped and/or balloon-shaped closure section 7, in this case, the second end section 8 of the implant does not remain in the longitudinal section 2 until the entire inverted section 4 is rolled out/expanded.

According to another embodiment of the invention, it can be provided that the region of the inverted section 4 delimiting the flow channel 5 comprises, at least in sections, means 9 for stiffening. Preferably, the means 9 for stiffening are foldings, preferably in the manner of an accordion, as is also illustrated in the figures. In this case, the length Lᵢ nevertheless merely describes the extent of the inverted section in the intended expansion direction, i.e. the length Lᵢ is identical for an inverted section 4 without foldings and an inverted section 4 with foldings.

In an alternative embodiment, the means 9 for stiffening can also be a reinforced section of the implant which is arranged such that at the time of insertion into the gastrointestinal tract, it forms the inverted section 4 in its entirety or forms a region thereof, i.e. the region delimiting the flow channel 5. For example, the reinforced section can be made of a different, stronger material than the remaining sections of the implant or of the same material but with a thicker wall.

Connection of the Implant to an Applicator

The implant is connected to an applicator 10 as described below:

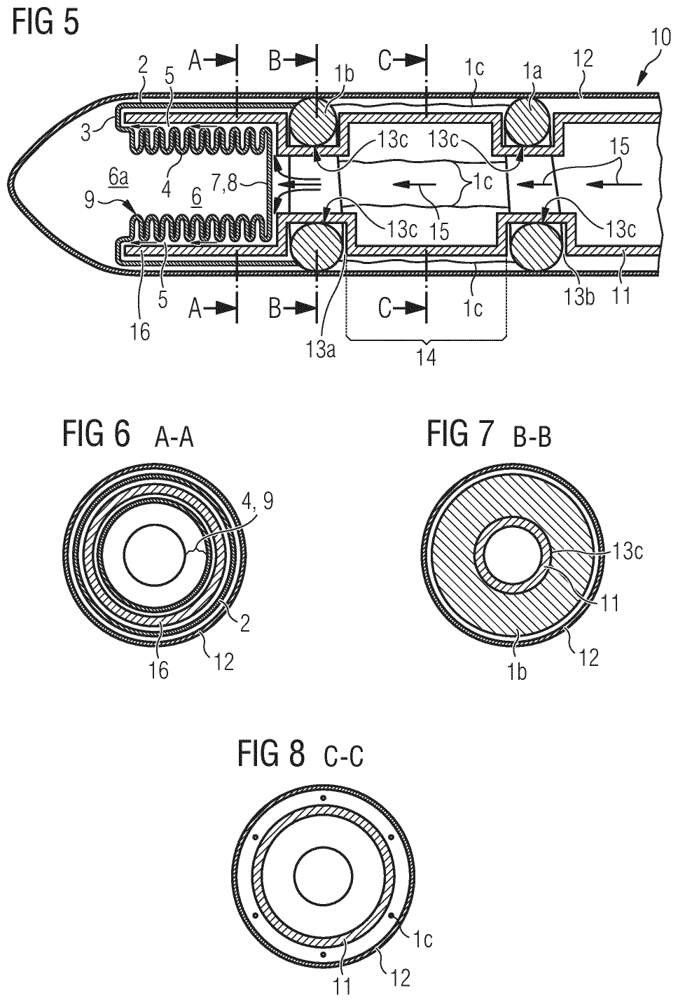

At the time of insertion into the gastrointestinal tract, the implant has one of the shapes illustrated in FIGS. 1a to 1d. Preferably, the implant is located at the front end of an applicator as illustrated in FIGS. 5, 9 and 10. Due to the different lengths L_{L} of the longitudinal section 2 or Lᵢ of the inverted section 4 at the time of insertion into the gastrointestinal tract, the embodiments of the implant shown in FIGS. 1a to 1d allow or require different connections of the implant to the applicator 10 by means of the securing elements 1a, 1b.

FIG. 5 shows an embodiment in which an implant according to FIG. 1a is connected to an applicator 10. However, the following explanations also apply analogously to the connection of an implant according to FIG. 1b or 1c to the applicator 10 shown in FIG. 5. The applicator 10 comprises an inner support body 11 and an outer sheath 12 which can be pulled off axially therefrom. Grooves 13a, 13b which are designed to receive the fastening elements 1a, 1b are located on the inner support body 11. The section 14 of the inner support body 11 extending between the fastening elements 1a, 1b is surrounded by the bands 1c connecting the fastening elements 1a, 1b, as is also illustrated in the sectional view in FIG. 8.

Both the inner support body 11 and outer sheath 12 are preferably mostly cylindrical in shape, with that of the inner support body 12 having a smaller diameter in the region of grooves 13a, 13b than in the other sections.

The diameter of the outer sheath 12 is selected such that it is in any case larger than the largest diameter of the inner support body 11. In this case, the oversize is also selected such that the substantially annular fastening elements 1a, 1b arranged in the grooves 13a, 13b are radially compressed. In FIGS. 5, 9 and 10, this is illustrated by cross-sections of the fastening elements 1a, 1b having different shapes.

Preferably, the fastening elements 1a, 1b also come into contact with the bottoms 13c of the grooves 13a, 13b, so that the securing elements 1a, 1b are clamped between the groove bottoms 13c and outer sheath 12, as illustrated in the sectional views according to FIGS. 5 and 7, so that they radially expand when the outer sheath 12 is axially pulled off the inner support body 11. However, radial expansion is also possible if the securing elements 1a, 1b do not come into contact with the groove bottoms 13c but only with the outer sheath 12.

However, it is principally also conceivable that the support body has no grooves, and the securing means 1a, 1b are nevertheless clamped between the support body 10 and the outer sheath 12.

The front-side end section of the applicator 10, which points in the insertion direction corresponding to the intended expansion direction E when the implant is inserted into the gastrointestinal tract, is formed as a cup-shaped holding section 16a which serves to receive the inverted section 4 of the implant and over which the longitudinal section 2 of the implant is slipped so that the cup-shaped holding section 16a is located inside the longitudinal section 2, as well as in the flow channel 5.

The inner support body 12 can be hollow or provided with an inner channel through which a fluid flow 15 can be introduced into the longitudinal section 2 to exert pressure on the closure section 7, which is flat in FIG. 5, and also on the transition section 3 via the flow channel 5.

In the exemplary embodiment according to FIG. 5, the inverted section 4 of the implant has means 9 for stiffening in the form of an accordion-like folding, which means allow the inverted section 4 to protrude into the interior of the holding section in a relatively stiff manner, forming a cavity 6. They also allow for a stable formation of the end section 7 as well as the orientation thereof substantially normal to the intended expansion direction E, to form an engagement surface for the fluid flow 15.

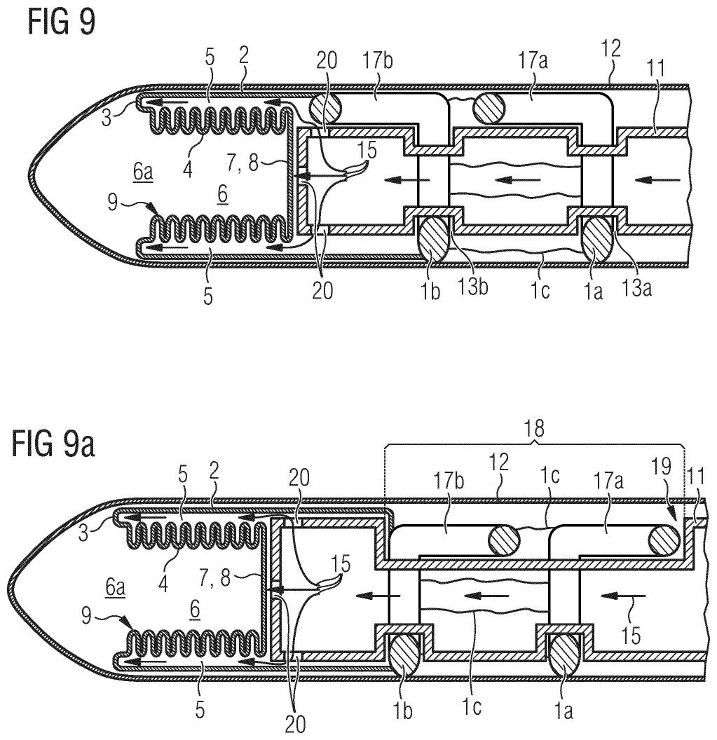

FIG. 9 shows an alternative connection possibility of the first embodiment of the implant according to the invention according to FIG. 1a to an applicator 10. However, the following explanations apply analogously also to the connection of an implant according to FIG. 1b or 1c to the applicator 10 shown in FIG. 9, which differs from that shown in FIG. 5 by a different front-side end region. In the exemplary embodiment shown in FIGS. 9 and 9a, the front-side end section 16b of the applicator 10, which faces in the insertion direction when the implant is inserted into the gastrointestinal tract, does not have a cup-shaped holding section, but rather ends bluntly, i.e. the end section 16b lying in the intended insertion direction does not offer any possibility of inverting/invaginating sections of the implant. Outlets 20 are provided distributed at the front-side end section 16b of the implant, through which the fluid can flow into the longitudinal section 2.

Due to the means for stiffening in the form of a folding 9, a support as provided by the wall of the cup-shaped holding section 16a of the inner support body 11 in the flow channel 5 (see FIG. 5) is not required. The longitudinal section 2, the transition section 3 as well as the inverted section 4 protrude from the applicator 10 in the intended expansion direction E, as illustrated in FIGS. 9 and 9a, even without additional support by the cup-shaped holding section 16a.

In addition, and independently of the formation of the front-side end portion of the applicator 10, FIG. 9 also illustrates that both of the fastening members 1a, 1b are folded down in a section 17a, 17b in the direction of the longitudinal axis of the inner support body 11, and the folded down sections 17a, 17b are clamped between the inner support body 11 and the outer sheath 12, while the sections that are not folded down are arranged extending in grooves 13a, 13b.

Folding down allows larger securing elements 1a, 1b (of larger diameter) to be used than is the case with the connection according to FIG. 5, since compression as shown there can only occur to a certain degree. Larger securing elements allow for greater expansion of the same and/or a smaller radial installation size of the applicator 10.

To further reduce the radial installation size of the applicator 10, it can further be provided that it has a reduced cross-section along a section 18 in which the folded down sections 17a, 17b rest against the inner support body 11, compared with the remaining sections, as illustrated in FIG. 9a. In this case, the sections 17a, 17b are arranged to lie completely in a recess 19 of the inner supporting body 11, so that they do not protrude beyond the circumference of the inner support body, unlike in the exemplary embodiment according to FIG. 9. The sections that are not folded down are arranged extending in grooves 13a, 13b.

In contrast to the exemplary embodiment according to FIG. 9, the folded down sections 17a, 17b of the fastening elements 1a, 1b in the exemplary embodiment according to FIG. 9a are furthermore folded down against the intended expansion direction E, which has advantages when folding up these sections for the purpose of engagement on the pylorus.

At this point, it should be noted that the individual distinguishing features shown in FIG. 5, FIGS. 9 and 9a can also be combined with each other. For example, it is conceivable that in the case of an applicator 10 with a cup-shaped holding section as shown in FIG. 5, the implant is connected by means of securing elements 1a, 1b with folded down sections 17a, 17b. For this purpose, the inner support body 11 can also have one or more sections 18 of reduced cross-section against which the folded down sections 17a, 17b can rest. The one or more sections 18 can form one or more recesses 19 which completely receives/receive the sections 17a, 17b so that they do not protrude beyond the circumference of the inner support body.

Regardless of the other features of the applicator 10, the folded down sections 17a, 17b can be either folded down in the intended expansion direction E or folded down against the intended expansion direction of the implant.

For example, the recess 19 described above can also be provided in combination with sections 17a, 17b folded down in the intended expansion direction E.

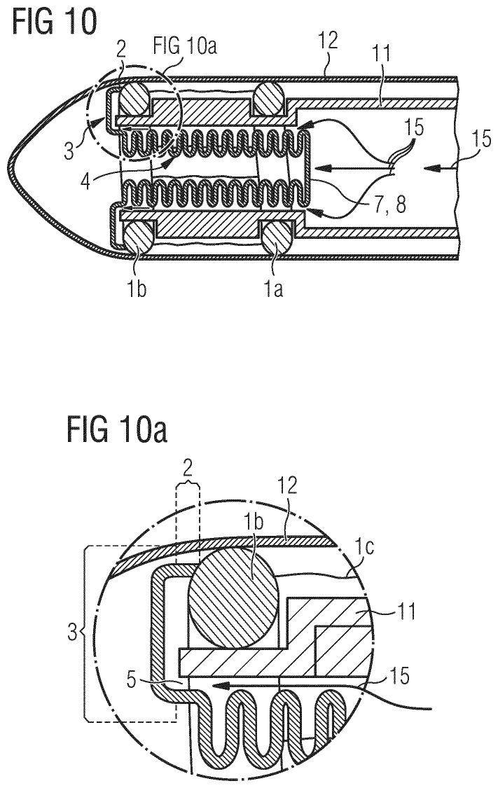

FIG. 10 shows a connection possibility for an implant according to the invention according to the fourth embodiment, as illustrated in FIG. 1d, to an applicator 10. This applicator is open at its front end pointing in the direction of insertion when the implant is inserted into the gastrointestinal tract.

Due to the short longitudinal section 2 at the time of insertion into the gastrointestinal tract and before the start of the expansion process, the implant with its one securing element 1b can be attached far forward on the applicator 10 as seen in the direction of insertion. This is followed by the longitudinal section 2 of the implant, which is only one or a few mm long, and then immediately by the transition section 3, which initiates the inversion of the implant, so that the entire inverted section 4 runs inside the inner support body 11.

Rolling Out the Implant

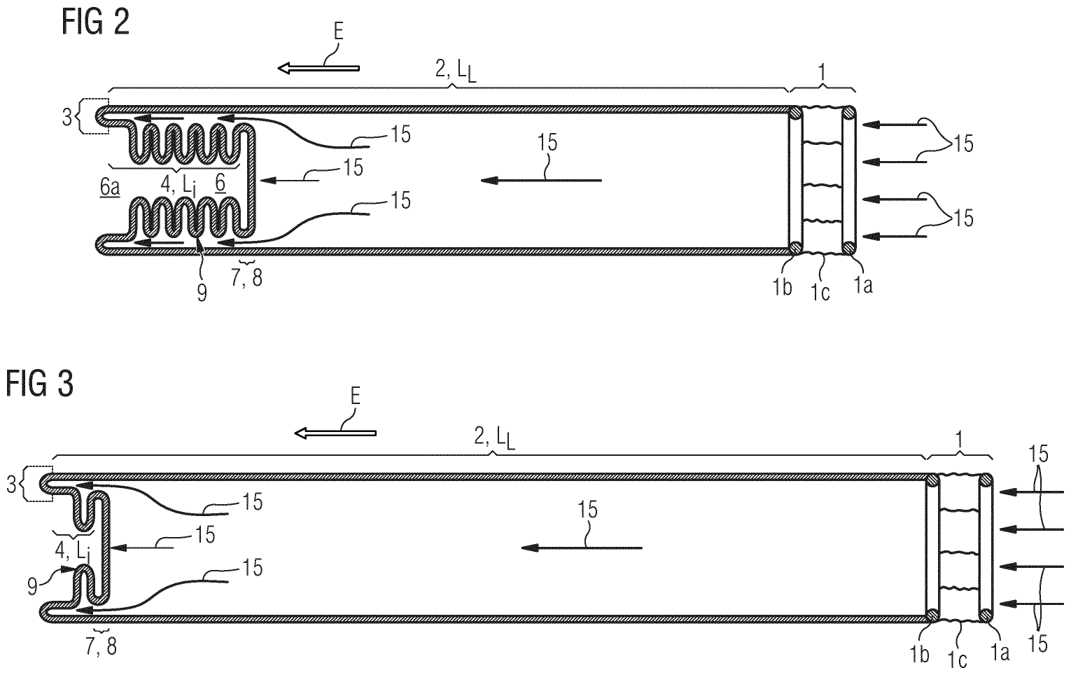

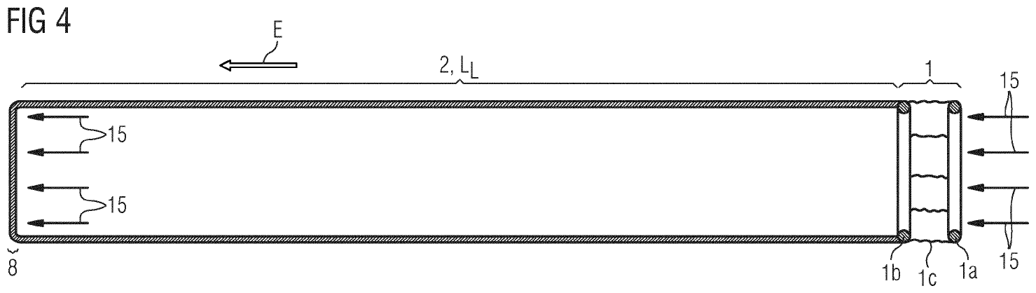

FIGS. 2 to 4 show the process of rolling out/expanding an implant according to the invention, with the expanded shape being illustrated expanded in a straight line for simplicity, whereas in practice, the expanded shape follows the convolutions of the intestinal tract.

The implant fixed to the applicator (not shown) is first conveyed to its position in the gastrointestinal tract by means of an endoscope (not shown) via the esophagus, and preferably in such a manner that one of the securing elements 1a is positioned on the gastric side of the pylorus and the other securing element 1b is positioned on the intestinal side. Preferably, however, it is provided that the implant fixed to the applicator 10 with its two securing elements 1a, 1b is also positioned in the gastrointestinal tract independently of the pylorus, and positioning in dependence on the pylorus is only carried out after the implant has fully expanded in the intestine.

Regardless of the manner of fixing the implant to the applicator shown in FIGS. 5 to 10, a fluid 15, preferably air, is subsequently blown into the longitudinal section 2 via the endoscope in a manner known per se. Preferably, blowing in the air into the tubular section of the implant takes place via the first end section 1 and a channel or cavity routed in the applicator 10, wherein even in the embodiment illustrated in FIG. 10, in which the inverted section 4 protrudes into the first end section 1 or protrudes through the first end section 1 against the intended expansion direction E, the fluid is regarded as being blown into the tubular section or longitudinal section 2, even though, viewed in the intended expansion direction E, the latter does not begin until after the securing element 1 and the fluid thus impinges almost directly on the transition section 3 (see FIG. 10a).

The blown in fluid 15 therefore flows in the intended expansion direction E first along the longitudinal section 2 and then, on the one hand, into the substantially annular flow channel 5 to the transition area 3 and, on the other hand, against the closure section 7 forming the second end section 8. In the embodiment according to FIG. 10/10a, the fluid 15 first impinges on the closure section 7 before flowing into the flow channel 5.

Subsequently, the pressure of the fluid 15 in the transition section 3 causes the inverted section 4 to roll out in the transition section 3, similar to the running gear of a tank but without running rollers. As a result, the length L_{L} of the longitudinal section 2 increases while the length Lᵢ of the inverted section 4 decreases at the same time.

Also, due to the fluid pressure acting on the closure section 7, the closure section is also continuously moved in the intended expansion direction E so that overall, due to the pressure of the fluid 15 in the transition section 3 and at the closure section 7, a relatively uniform rolling out of the implant into the intestinal tract takes place, as is shown schematically in FIGS. 2 and 3. By means of the pressure of the fluid 15 in the flow channel 5, pressure is also exerted on the longitudinal section 2 so that it can rest against the intestinal wall. The cavity 6 supports the rolling out of the inverted section 4 in the transition area 3 in that the outward inversion of the inverted section 4 into this section is not slowed down or even blocked by the inverted section 4 rubbing against itself.

It should be noted that in FIGS. 2 and 3 the flow channel 5 is always shown open. As already explained, it cannot be ruled out that the region of the inverted section 4 delimiting the flow channel 5 makes contact in sections in a releasable manner with the region of the longitudinal section 2 delimiting the flow channel 5 or vice versa, but this is not shown separately in the drawings.

In this case, these regions are forced apart by the fluid flow 15 and the contacting is released so that the fluid 15 can advance to the transition area 3.

In the embodiment of an implant shown in the figures, the region of the inverted section 4 delimiting the flow channel 5 has means 9 for stiffening in the form of foldings running in an accordion-like manner. These help to ensure that the inverted section is and remains relatively dimensionally stable running in the intended expansion direction E, so that the implant according to the invention protrudes from the applicator 10. Since the longitudinal section 2 surrounding the inverted section 4 in the embodiment shown, although not excluded in principle, has no means 9 for stiffening, it is to be expected, particularly in the embodiment shown in FIGS. 2 and 3, that regions of the inverted section 4 and regions of the longitudinal section 2 will come into contact with each other and that this contacting will be released by the fluid flow 15.

Similarly, when inserting the implant into the gastrointestinal tract, coming into contact with the esophagus, stomach, pylorus, or intestines can result in an external application of force on the longitudinal section 2 such that the longitudinal section is pressed against and makes contact with the inverted section 2. In this case, the pressure with which the fluid 15 is to be blown in into the longitudinal section 2 is to be selected to be correspondingly high, so that this contacting is released again and the fluid 15 can advance to the transition section 3.

In those cases in which the closure section 7 is closed after the implant has been rolled out in the intestinal tract, it must be opened. This can be done in a manner known per se, for example by making the closure section 7 of a material which dissolves due to digestive juices after a predeterminable period of time, or by means of a perforation in the region of the closure section 7 which, when the implant is completely rolled out, detaches the end section 7 from the rest of the implant due to the fluid pressure and thus removes the second end section 8 of the implant.

Subsequently, also in a known manner, the intestinal-side securing element 1b can then be positioned on the intestinal side with respect to the pylorus and the outer sheath 12 can be retracted towards the stomach to such an extent that initially only the intestinal-side securing element 1b expands and/or its folded down section 1a is folded up or can fold up and rests on the intestinal side against the pylorus.

Subsequently, the endoscope including the applicator 10, with the securing element 1a still clamped between the inner support body 11 and the outer sheath 12, is moved towards the stomach until it is ensured that the securing element 1a is located on the stomach side of the pylorus. Only then will the outer sheath 12 be retracted further towards the stomach so that the securing element 1a can also expand and/or its folded down section 17a folds up/can fold up.

For the sake of completeness, it should be mentioned here that in order to be able to retract the outer sheath 12 towards the stomach, its intestinal-side end portion 12a must first be removed. This is possible in a manner known per se, analogous to the removal of the end section 7,8 described above.

### REFERENCE LIST

1 first end section of the implant
1a first annular securing element
1b second annular securing element
1c elastic bands
2 longitudinal section of the implant
3 transition section of the implant
4 inverted section of the implant
5 flow channel
6 cavity
6a cavity opening
7 closure section
8 second end section of the implant
9 means for stiffening the inverted section
10 applicator
11 inner support body
12 outer sheath
12a intestinal-side end portion of outer sheath
13a groove for receiving a securing element
13b groove for receiving a securing element
13c groove bottom
14 section of inner support body
15 fluid/fluid flow
16a cup-shaped holding section
16b blunt end section
17a folded down section of a securing element
17b folded down section of a securing element
18 section of the inner support body 11 for engagement of 17a, 17b
19 recess on the inner support body
20 outlet

1. An implant for arranging and expanding in the human intestinal tract for the purpose of lining a section of the intestinal tract in order to prevent resorption of supplied nourishment in said section, having
a first end section (1) which, as a securing section, allows the fixation of the implant in the human gastrointestinal tract and/or to an applicator (10),
a tubular section adjoining the first end section (1), comprising
a longitudinal portion (2) extending in the intended expansion direction, and
a transition portion (3) adjoining the longitudinal portion (2), and
an inverted section (4) running within the longitudinal section in the opposite direction to the intended expansion direction, and
a second end portion (8),
characterized in that
a region of the inverted section (4) and a region of the longitudinal section delimit a flow channel (5) via which a fluid (15) blown into the tubular section can penetrate to the transition section (3) in order to exert pressure thereon, wherein the inverted section (4) radially delimits a cavity (6) and has a closure section (7) which forms the second end section (8) and axially delimits the cavity (6).

2. The implant according to claim 1, characterized in that the closure section (7) is formed flat.

3. The implant according to claim 1, characterized in that the closure section (7) is created by gathering a region of the inverted section (4) distant from the transition section (3).

4. The implant according to any one of the preceding claims, characterized in that the closure section (7) is arranged within the longitudinal section (2).

5. The implant according to any one of the preceding claims, characterized in that the region of the inverted section (4) delimiting the flow channel (5) has means for stiffening (9) at least in sections.

6. The implant according to claim 5, characterized in that the means for stiffening (9) are foldings.

7. The implant according to claim 5 or 6, characterized in that the region of the inverted section (4) delimiting the flow channel (5) comes into contact in sections in a releasable manner with the region of the longitudinal section (2) delimiting the flow channel (5).

8. The implant according to claim 5 or 6, characterized in that the region of the inverted section (4) delimiting the flow channel (5) runs spaced apart from the region of the longitudinal section (2) delimiting the flow channel (5).

9. The implant according to any one of the preceding claims, characterized in that the inverted section (4) is shorter than the longitudinal section (2).

10. The implant according to any one of claims 3 to 9, characterized in that the closure portion (7) is open.

11. The implant according to any one of claims 1 or 2 or 5 to 10, characterized in that the closure section (7) is cup-shaped and/or balloon-shaped and protrudes into or out of the cavity (6) in the intended expansion direction (E).
